(19) United States
(12) Patent Application Publication
(10) Pub. No.: US 2011/0318296 A1
(43) Pub. Date: Dec. 29, 2011

(54) SOLID AIR FRESHENER

(75) Inventors: Norbert Andreas Braun, Singapore (SG); Tee Yong Tan, Singapore (SG)

(73) Assignee: SYMRISE GmbH & Co. KG, Holzminden (DE)

(21) Appl. No.: 12/668,969

(22) PCT Filed: May 14, 2007
(86) PCT No.: PCT/EP2007/054666

§ 371 Date: Apr. 10, 2010

(51) Int. Cl. A61L 9/01; B29C 39/38

(52) U.S. Cl. 424/76.1; 422/120; 264/299; 264/319

(57) ABSTRACT

The present invention relates to a solid air freshener comprising the following components: i) a total amount of polyethylene glycols in the range of from 30 to 80 Wt.-%, wherein the weight average molecular weight M_{w} of the total amount of polyethylene glycols present in the solid air freshener is in the range of from 1000 to 20000 g/mol, the total amount of polyethylene glycols consisting of one, two, or more polyethylene glycol materials, ii) a total amount of surfactants in the range of from 5 to 40 wt.-%, iii) a total amount of perfume in the range of from 5 to 50 wt.-%, wherein the perfume consists of (iii)(a) fragrance material, and optionally (iii)(b) a solvent for the fragrance material, iv) an amount of water in the range of from 0 to 10 wt.-%, wherein any water present in the solid air freshener is exclusively considered as component (iv), and wherein the total amount of components (i), (ii), (iii), and (iv) is at least 95 wt.-%, based on the total weight of the solid air freshener.

The present invention relates to a solid (at 20° C. and 1013 mbar) air freshener particularly suitable for use in a humid and warm environment, such as a shower or a bathroom. A receptacle comprising a (bathroom) air freshener according to the present invention whereby water can be collected and its outflow regulated to give a perceivable burst of fragrance is also described.

### TECHNICAL FIELD

In the natural course of a shower or a bath, a lot of water is used and discharged through the drain. This waste water can be utilized by the invention to provide a pleasant (air freshening) fragrance burst, e.g. in the shower cubicle or bathroom.

### BACKGROUND & PRIOR ART

The majority of conventional solid air freshening articles allow the fragrance to diffuse in a consistent manner from a static position. The consistent diffusion of the fragrance does not have a surprise element to the consumer, however. At the end of such an article's life-cycle, there is often a residue that requires disposal. This is inconvenient for the consumer and unfavourable in terms of waste generation.

Traditional solid forms of air freshening articles are typically composed of gel-forming agents, surfactants, perfume and a volatile liquid (water/alcohol combinations etc). These articles leave a residue after the evaporation of the volatile liquid carrier, and the presence of water limits the amount of fragrance that can be incorporated into the system. In water-based systems such as systems comprising a carrageenan gel, the amount of fragrance incorporated requires a corresponding amount of surfactants and/or surfactant/alcohol combinations to keep the fragrance adequately solubilized in the respective system. The typical amount of fragrance incorporated usually does not exceed 20 wt.-% of the total weight of the system. At the end of the life-cycle, the gel-forming agents and the surfactants remain as a residue for disposal.

Many forms of solid air freshening articles have been developed and are usually based on the mechanism of static diffusion. The air freshening article is placed in a fixed location representing the source of fragrance diffusion.

U.S. Pat. No. 4,904,639 teaches the use of polyethylene glycol, water and hydrophobic perfume as an air freshening system. The separation of perfume away from the hydrophilic phase onto the surface of the system promotes the diffusion of the perfume into the air. The presence of water in the product according to U.S. Pat. No. 4,904,639 limits the amount of perfume, e.g. in the example cited the amount of perfume is less than 20 wt.-%. A system based on the immiscibility of the perfume and water causes problems regarding the rate of diffusion as the migration of the perfume out of the system is difficult to control. The perfume may migrate completely onto the surface of the block before the consumer has a chance to open the package resulting in an unpleasant “wet” appearance of the air freshener block. In this case there would also be a strong diffusion of the perfume in the initial stages of use as most of the perfume is located on the surface of the block. Once the perfume on the surface has completely diffused, the performance of the system becomes weak.

### DESCRIPTION OF INVENTION

It was the object of the present invention to provide a solid air freshener minimizing the drawbacks mentioned above. In particular, the solid air freshener should be able to provide a strong burst of fragrance, which may have a surprise element to the consumer. The air freshener should not leave a residue for disposal at the end of its life-cycle. In addition, the amount of fragrance that can be incorporated into the system should be larger than in case of conventional solid air fresheners. It is also desirable that the fragrance is released from a large area rather than from a static location.

The invention is based on the surprising finding that this object can be achieved by a solid air freshener comprising the following components:
i) a total amount of polyethylene glycols in the range of from 30 to 80 wt.-%,
   wherein the weight average molecular weight M_{W} of the total amount of polyethylene glycols present in the solid air freshener is in the range of from 1000 to 20000 g/mol, the total amount of polyethylene glycols consisting of one, two, or more polyethylene glycol materials,
ii) a total amount of surfactants in the range of from 5 to 40 wt.-%,
iii) a total amount of perfume in the range of from 5 to 50 wt.-%, wherein the perfume consists of
   (iii)(a) fragrance material, and optionally
   (iii)(b) a solvent for the fragrance material,
iv) an amount of water in the range of from 0 to 10 wt.-%,
   wherein any water present in the solid air freshener is exclusively considered as component (iv), and
   wherein the total amount of components (i), (ii), (iii), and (iv) is at least 95 wt.-%, based on the total weight of the solid air freshener.

With respect to the assignment of constituents to components (i), (ii), (iii), and (iv), it has to be noted that in certain cases, a constituent may be assigned to more than one group. In this case, the following applies:
If a constituent can be assigned to more than one of the components (i), (ii), (iii), it is assigned to the first of the mentioned components to which it can be assigned.

For example, if a constituent is both a surfactant (component (ii)) and a solvent for the fragrance material (component (iii), it is assigned to component (ii)). If a polyethylene glycol (component (i)) is also a solvent for the fragrance material (component (iii)), it is assigned to component (i).
However, a number of commonly used solvents for fragrance materials, namely ethanol, isopropanol, diethylene glycol monoethyl ether, glycerol, propylene glycol, 1,2-butylene glycol, dipropylene glycol, diethyl phthalate, triethyl citrate, and isopropyl myristate, are always assigned to component (iii).
Any water is always assigned to component (iv).

The air freshener according to the present invention is virtually completely water-soluble and produces a burst of fragrance when contacted with (preferably warm) water, e.g. when taking a shower.

The basic principle of evaporation is that the larger the surface area, the faster the rate of evaporation. In the present invention, water dissolving the surface of the solid air freshener (solid air freshening article) promotes release (evaporation) of the fragrance from the matrix, and the diffusion of the fragrance is enhanced over a large surface area. The situation of a consumer taking a shower or a bath is a typical example of a situation for which applications based on this principle of evaporation can be developed.

The water-soluble matrix in contact with water releases an amount of fragrance which is volatized into the air as the waste water travels the distance to the drainage system. The increased surface area allows the diffusion of the fragrance and gives the consumer a perceivable sensation of a strong burst of fragrance. The rate of diffusion can be adjusted by the water solubility of the matrix as well as the receptacle device regulating the amount of water collected and its rate of discharge from the receptacle. At the end of its life-cycle, there is no residue for disposal.

The present invention uses a novel approach to the field of air freshening devices as an alternative to static diffusion of perfume from a solid air freshening article. The present invention shows that it is possible to develop a solid air freshening article that provides air freshening properties over a large area rather than from a static location and does not leave a residue for disposal at the end of its life-cycle.

The water solubility of the solid air freshener can be controlled by the amount of polyethylene glycol(s) and surfactant(s). In addition, the range of the molecular weight of polyethylene glycol can also be an important factor as a polyethylene glycol with a molecular weight in the lower range such as 1500 is more easily water-soluble than a polyethylene glycol with a molecular weight of 9000. The hardness of the article can also be controlled by the type of polyethylene glycol used. A polyethylene glycol with a molecular weight of 9000 can provide more hardness to the system that a polyethylene glycol with a molecular weight of 1500. The amount of surfactants used can also adjust the water solubility of the system. For example within the class of nonionic surfactants, a higher-ethoxylated surfactant has better water solubility properties than a lower-ethoxylated surfactant.

The rate of fragrance diffusion in the air freshener according to the invention is dependent on the water solubility of the system as well as on the surface area for evaporation. The invention also contemplates the rate whereby the fragrance released from the matrix is distributed across the surface for evaporation. In the setting of a shower cubicle, when the consumer takes a shower, water is collected in the receptacle containing the air freshening article (air freshener). This collected water dissolves part of the surface of the article, releases the fragrance from the surface and is allowed to flow out of the receptacle towards the drainage system in the shower cubicle. The number and size of the discharge outlets in the receptacle can exert a control on the flow rate of the water containing or releasing the fragrance. The presence of viscosity-increasing agents in the solid air freshener such as cationic polymers or film-formers can affect the speed at which the waste water flows towards the drainage system. The use of viscosity-increasing agents is particularly desirable if the shower cubicle is small and the distance from the receptacle to the drainage system is short since it will help to retard the rate of flow and allow more time for the diffusion of the fragrance. Cationic surfactants with an affinity for hard surfaces can also be useful in the invention, and are a preferred ingredient of an inventive solid air freshener.

In the setting of a bath-tub, the inventive solid air freshener can be fixed near the tap and also be a carrier for agents like e.g. skin-conditioning agents to be released into the bath water.

In both situations, the consumer can experience an (air freshening) burst of fragrance in an enclosed area. As an example for fragrances, aroma-therapeutic fragrances can give a pleasant experience to the consumer.

In a certain embodiment, a receptacle for an inventive solid air freshener is designed with a collecting plate for the fragrance-containing water. An example would be a two-tier device whereby water can be collected and discharged from the receptacle containing the air freshener onto a collecting plate or trough for the process of evaporation and diffusion of the fragrance.

Preferred is a solid air freshener according to the invention, wherein

(i) the total amount of polyethylene glycols is in the range of from 40 to 70 wt.-%,
and/or
(ii) the total amount of surfactants in the range of from 10 to 30 wt.-%,
and/or
(iii) the total amount of perfume is in the range of from 5 to 40 wt.-%, preferably 10 to 30 wt.-%
and/or
(iv) the amount of water is in the range of from 0 to 5 wt.-%,
wherein the total amount of components (i), (ii), (iii), and (iv) is at least 95 wt.-%, based on the total weight of the solid air freshener.

Particularly preferred is a solid air freshener according to the invention, wherein

(i) the total amount of polyethylene glycols is in the range of from 40 to 70 wt.-%,
and
(ii) the total amount of surfactants in the range of from 10 to 30 wt.-%,
and
(iii) the total amount of perfume is in the range of from 10 to 30 wt.-%,
and
(iv) the amount of water is in the range of from 0 to 5 wt.-%,
wherein the total amount of components (i), (ii), (iii), and (iv) is at least 95 wt.-%, based on the total weight of the solid air freshener.

In contrast to the air fresheners disclosed in U.S. Pat. No. 4,904,639 the air fresheners according to the present invention have a significant lower water content, i.e. 0 to 10 wt.-%, preferably 0 to 5 wt.-%, more preferred only 0 to 2 wt.-%, based on the total weight of the solid air freshener.

### Polyethylene Glycols (Component i))

Polyethylene glycols (PG), preferably solid at room temperature (20° C.) with a weight average molecular weight (M_{W}) of 1500 and above, can be obtained from BASF (Pluriol®), Clariant (Polyglykol®), Dow (Carbowax™) and various other suppliers. The polyethylene glycols suitable for the present invention have a (weight average) molecular weight in the range of 1000-20000, preferably in the range of 1500-9000. Polyethylene glycols with higher molecular weight (M_{W}), in particular those having a M_{W} greater than 12000, are less preferred because of their higher melting point, which inter alia is disadvantageous to the fragrance materials to be incorporated in the air fresheners of the present invention.

Air fresheners according to the present invention having a TMWFL-index of at least 7000 are preferred, preferably in the range of 8000-25000, more preferably in the range of 9000-15000. The TMWFL-index herein defined is the ratio of the “total molecular weight” (=wt.-% of each polyethylene glycol used times respective molecular weight) of the polyethylene glycols used divided by the total amount of fragrance materials (component (iii)(a), i.e. fragrance load) used. Such air fresheners have been found to possess the desired performance and balance in terms of hardness, fragrance diffusion, water-dissolution and wear-rate.

Throughout this text, the term “polyethylene glycol material” refers to a polydisperse group of polyethylene glycol molecules possessing a single peak in their molecular weight distribution.

In certain cases, solid air fresheners are desired wherein component (i) consists of a single polyethylene glycol material. In these cases a solid air freshener according to the invention is preferred, wherein component i) consists of a single polyethylene glycol material having a weight average molecular weight M_{W} in the range of from 4000 to 9000. Particularly preferred is such a solid air freshener, wherein the ratio (TMWFL Index)

[(M_{W(i)})×weight percentage of component (i)]:[(weight percentage of component (iii)(a))×g/mol]

is greater than 7000, preferably in the range of from 8000 to 25000, more preferably in the range of from 9000 to 15000.

It is however often preferred to use mixtures of two or more polyethylene glycol materials. Preferred is a solid air freshener according to the invention, wherein component i) consists of

(i)(a) a first polyethylene glycol material having a weight average molecular weight M_{W(i)(a)} in the range of from 1500 to 3000, and
(i)(b) a second polyethylene glycol material having a weight average molecular weight M_{W(i)(b)} in the range of from 5500 to 9500, and
optionally one or more further polyethylene glycol materials having a different weight average molecular weight M_{W}.

Such mixtures have shown to have a good hardness, fragrance diffusion, water-dissolution and wear-rate. It is often preferred to use no more than two polyethylene glycol materials as described above.

Particularly preferred is a solid air freshener according to the invention wherein, component i) consists of or comprises

(i)(a) a first polyethylene glycol material having a weight average molecular weight M_{W(i)(a)} in the range of from 1500 to 3000, and
(i)(b) a second polyethylene glycol material having a weight average molecular weight M_{W(i)(b)} in the range of from 5500 to 9500,
wherein the ratio (TMWFL Index)

[(M_{W(i)(a)}×weight percentage of component (i)(a))+(M_{W(i)(b)}×weight percentage of component (i)(b))]:[(weight percentage of component (iii)(a))×g/mol]

is greater than 7000. Particularly preferred is a solid air freshener, wherein said ratio is in the range of from 8000 to 25000, preferably in the range of from 9000 to 15000.

In case component (i) consists of two ore more polyethylene glycol materials, it is preferred that in the solid air freshener according to the invention each of said materials has a weight average molecular weight M_{W} in the range of from 1000 to 20000, two or more polyethylene glycol materials having a difference between the respective weight average molecular weights of at least 2000, preferably of at least 3000.

In contrast to the air freshener according to the present invention, the air freshener of U.S. Pat. No. 4,904,639 did not harden properly and a separation of fragrance materials was observed on the surface of the air freshener according to U.S. Pat. No. 4,904,639 in own experiments.

| | PG | PG | PG | | | | |
|---|---|---|---|---|---|---|---|
| | M.W. | M.W. | M.W. | PG | PG | | |
| | 1500 | 2000 | 4000 | M.W. | M.W. | | |
| | wt.- | wt.- | wt.- | 6000 | 9000 | Perfume | TMWFL- |
| Example | % | % | % | wt.-% | wt.-% | wt.-% | index |
| A | 30 | — | — | — | 20 | 20 | 11250 |
| B | — | — | — | — | 40 | 50 | 7200 |
| C | — | — | 50 | — | — | 20 | 10000 |
| D | — | 20 | — | 30 | — | 20 | 11000 |
| E | 10 | — | — | — | 40 | 40 | 9375 |

### Exemplary Calculation of the TMWFL-Index in Case of Example A:

$$\begin{matrix}PG M . W . 1500 \times 30 wt . \text{-} \% & = 45000 \\ PG M . W . 9000 \times 20 wt . \text{-} \% & = 180000 \\ Total & = 225000\end{matrix}$$

$$TMWFL \text{-} index \text{:} 225000 / 20 wt . \text{-} \% perfume = 11250$$

Preferred Polyethylene Glycols for use in solid air fresheners according to the invention are selected from the following (e.g. Polyglykol® from Clariant and Pluriol® E from BASF).

| | | | | Water | Hydroxyl |
|---|---|---|---|---|---|
| | | Molar | Solidification | content | value |
| | Product | mass | Point | (DIN 51777) | (DIN 53240) |
| Type | form | g/mol | EP III ° C. | % (m/m) | mg KOH/g |
| Polyglykol | Wax/melt | 950-1,050 | 35-40 | max. 0.5 | 107-118 |
| 1000 | | | | | |
| Polyglykol | Wax/melt | 950-1,050 | 35-40 | max. 0.15 | 107-118 |
| 1000 WA | | | | | |
| Polyglykol | Flakes | 1,400-1,600 | 44-48 | max. 0.5 | 70-80 |
| 1500 S | | | | | |
| Polyglykol | Melt | 1,400-1,500 | 44-48 | max. 0.1 | 70-80 |
| 1500 FL | | | | | |
| Polyglykol | Flakes | 1,800-2,200 | 48-52 | max. 0.5 | 51-62 |
| 2000 S | | | | | |
| Polyglykol | Melt | 1,800-2,200 | 48-52 | max. 0.1 | 51-62 |
| 2000 FL | | | | | |
| Polyglykol | Flakes | 2,700-3,300 | 52-56 | max. 0.5 | 34-42 |
| 3000 S | | | | | |
| Polyglykol | Melt | 2,700-3,300 | 52-56 | max. 0.1 | 34-42 |
| 3000 FL | | | | | |
| Polyglykol | Flakes*)/**) | 3,050-3,700 | 53-57 | max. 0.5 | 30-37 |
| 3350 S | | | | | |
| Polyglykol | Melt | 3,050-3,700 | 53-57 | max. 0.1 | 30-37 |
| 3350 FL | | | | | |
| Polyglykol | Flakes*)/**) | 3,700-4,400 | 53-58 | max. 0.5 | 25-30 |
| 4000 S | | | | | |
| Polyglykol | Wax/melt | 3,700-4,400 | 53-58 | max. 0.1 | 25-30 |
| 4000 FL | | | | | |
| Polyglykol | Wax/melt | 5,100-5,900 | 55-60 | max. 0.1 | 19-22 |
| 5500 FL | | | | | |
| Polyglykol | Flakes*)/**) | 5,600-6,600 | 55-60 | max. 0.5 | 17-20 |
| 6000 S | | | | | |
| Polyglykol | Wax/melt | 5,600-6,600 | 55-60 | max. 0.1 | 17-20 |
| 6000 FL | | | | | |
| Polyglykol | Flakes*)/**) | 7,000-9,000 | 55-60 | max. 0.5 | 12-16 |
| 8000 S | | | | | |
| Polyglykol | Wax/Melt | 7,000-9,000 | 55-60 | max. 0.1 | 12-16 |
| 8000 FL | | | | | |

*)These products are also available in form of a powder.
**)These products are also available in form of a fine powder.

| | | | Viscosity | |
|---|---|---|---|---|
| | Product | Molar mass | at 75° C. | Melting |
| Type | form | g/mol | [mm²/s] | Point ° C. |
| Pluriol E 1000 | Paste | approx. 1000 | approx. 30 | approx. 40 |
| Pluriol E 1500 E | Solid | approx. 1500 | approx. 60 | approx. 45 |
| Pluriol E 1500 | Powder | approx. 1500 | approx. 60 | approx. 45 |
| Powder | | | | |
| Pluriol E 1500 | Flakes | approx. 1500 | approx. 60 | approx. 45 |
| Flakes | | | | |
| Pluriol E 3400 E | Solid | approx. 3400 | approx. 200 | approx. 55 |
| Pluriol E 3400 | Powder | approx. 3400 | approx. 200 | approx. 55 |
| Powder | | | | |
| Pluriol E 3400 | Flakes | approx. 3400 | approx. 200 | approx. 55 |
| Flakes | | | | |
| Pluriol E 4000 E | Solid | approx. 4000 | approx. 260 | approx. 55 |
| Pluriol E 4000 | Powder | approx. 4000 | approx. 260 | approx. 55 |
| Powder | | | | |
| Pluriol E 4000 | Flakes | approx. 4000 | approx. 260 | approx. 55 |
| Flakes | | | | |
| Pluriol E 6000 E | Solid | approx. 6000 | approx. 600 | approx. 60 |
| Pluriol E 6000 | Powder | approx. 6000 | approx. 600 | approx. 60 |
| Powder | | | | |
| Pluriol E 6000 | Flakes | approx. 6000 | approx. 600 | approx. 60 |
| Flakes | | | | |
| Pluriol E 8000 E | Solid | approx. 8000 | approx. 1500 | approx. 63 |
| Pluriol E 8000 | Flakes | approx. 8000 | approx. 1500 | approx. 63 |
| Flakes | | | | |
| Pluriol E 9000 | Powder | approx. 9000 | approx. 2500 | approx. 65 |
| Powder | | | | |
| Pluriol E 9000 | Flakes | approx. 9000 | approx. 2500 | approx. 65 |
| Flakes | | | | |

Viscosity: measured using an Ubbelohde viscosity meter according to DIN 51562 [mm²/s]
Molar mass calculated from hydroxyl number according to DIN 53240 or PSA method

Surfactants (Component ii))

Surfactants, preferably nonionic surfactants with an ethoxylated value of 10 and above, can be obtained from Cognis (Eumulgin®), BASF (Lutensol®), Croda (Volpo®), Shell Chemicals (Neodol®) and various suppliers.

Surfactants are a desirable component to confer a degree of water solubility to the system. The surfactants of component ii) of an air freshener according to the present invention are substances that are preferably solid at 20° C. Preferred surfactants are solid nonionic surfactants, fatty alcohol ethoxylates, preferably of C₁₄-C₁₈ fatty alcohols, preferably with 10 ethoxylated units or more, further preferred having 11-80 ethoxylated units, because these fatty alcohol ethoxylates help to adjust the dissolution rate (in water) of the air fresheners according to the present invention.

The following ethoxylated stearyl alcohols are preferred:

polyethylene glycol (n) stearyl ether (steareth-n), where n=13-50,
polyethylene glycol (n) cetyl ether (ceteth-n), where n=13-30,
polyethylene glycol (n) isocetyl ether (isoceteth-n), where n=13-30,
polyethylene glycol (n) cetylstearyl ether (ceteareth-n), where n=11-80, preferred where n=18-50,
polyethylene glycol (m) isostearyl ether (isosteareth-m), where m=12-20
polyethylene glycol (k) oleyl ether (oleth-k), where k=12-15

It is advantageous to select the fatty alcohol ethoxylates from the group comprising ethoxylated stearyl alcohols, cetyl alcohols and cetylstearyl alcohols (cetearyl alcohols). The following are particularly preferred:

polyethylene glycol (13) stearyl ether (steareth-13), polyethylene glycol (14) stearyl ether (steareth-14), polyethylene glycol (15) stearyl ether (steareth-15), polyethylene glycol (16) stearyl ether (steareth-16), polyethylene glycol (17) stearyl ether (steareth-17), polyethylene glycol (18) stearyl ether (steareth-18), polyethylene glycol (19) stearyl ether (steareth-19), polyethylene glycol (20) stearyl ether (steareth-20), polyethylene glycol (21) stearyl ether (steareth-21), polyethylene glycol (30) stearyl ether (steareth-30), polyethylene glycol (12) isostearyl ether (isosteareth-12), polyethylene glycol (13) isostearyl ether (isosteareth-13), polyethylene glycol (14) isostearyl ether (isosteareth-14), polyethylene glycol (15) isostearyl ether (isosteareth-15), polyethylene glycol (16) isostearyl ether (isosteareth-16), polyethylene glycol (17) isostearyl ether (isosteareth-17), polyethylene glycol (18) isostearyl ether (isosteareth-18), polyethylene glycol (19) isostearyl ether (isosteareth-19), polyethylene glycol (20) isostearyl ether (isosteareth-20), polyethylene glycol (13) cetyl ether (ceteth-13), polyethylene glycol (14) cetyl ether (ceteth-14), polyethylene glycol (15) cetyl ether (ceteth-15), polyethylene glycol (16) cetyl ether (ceteth-16), polyethylene glycol (17) cetyl ether (ceteth-17), polyethylene glycol (18) cetyl ether (ceteth-18), polyethylene glycol (19) cetyl ether (ceteth-19), polyethylene glycol (20) cetyl ether (ceteth-20), polyethylene glycol (13) isocetyl ether (isoceteth-13), polyethylene glycol (14) isocetyl ether (isoceteth-14), polyethylene glycol (15) isocetyl ether (isoceteth-15), polyethylene glycol (16) isocetyl ether (isoceteth-16), polyethylene glycol (17) isocetyl ether (isoceteth-17), polyethylene glycol (18) isocetyl ether (isoceteth-18), polyethylene glycol (19) isocetyl ether (isoceteth-19), polyethylene glycol (20) isocetyl ether (isoceteth-20), polyethylene glycol (12) oleyl ether (oleth-12), polyethylene glycol (13) oleyl ether (oleth-13), polyethylene glycol (14) oleyl ether (oleth-14), polyethylene glycol (15) oleyl ether (oleth-15), polyethylene glycol (12) lauryl ether (laureth-12), polyethylene glycol (12) isolauryl ether (isolaureth-12), polyethylene glycol (13) cetylstearyl ether (ceteareth-13), polyethylene glycol (14) cetylstearyl ether (ceteareth-14), polyethylene glycol (15) cetylstearyl ether (ceteareth-15), polyethylene glycol (16) cetylstearyl ether (ceteareth-16), polyethylene glycol (17) cetylstearyl ether (ceteareth-17), polyethylene glycol (18) cetylstearyl ether (ceteareth-18), polyethylene glycol (19) cetylstearyl ether (ceteareth-19), polyethylene glycol (20) cetylstearyl ether (ceteareth-20) and polyethylene glycol (30) cetylstearyl ether (ceteareth-30).

Preferred are steareth-21, ceteareth-12, ceteareth-20 and ceteareth-30 and mixtures thereof.

Perfume (Component iii))

Perfumes in the context of the present invention can be single fragrance substances (perfumery ingredients, fragrance materials) or mixtures thereof (perfume oils, mixtures of perfumery ingredients). Perfumes used in the present invention can be selected from the large palette of perfumes available for creative perfumery work.

Preferred extracts of natural raw materials such as essential oils, concretes, absolutes, resins, resinoids, balsams, tinctures are selected from the group consisting of:

ambergris tincture; amyris oil; angelica seed oil; angelica root oil; aniseed oil; valerian oil; basil oil; wood moss absolute; bay oil; mugwort oil; benzoin resin; bergamot oil; beeswax absolute; birch tar oil; bitter almond oil; savory oil; bucco-leaf oil; cabreuva oil; cade oil; calamus oil; camphor oil; cananga oil; cardamom oil; cascarilla oil; cassia oil; cassia absolute; castoreum absolute; cedar-leaf oil; cedarwood oil; cistus oil; citronella oil; lemon oil; copaiba balsam; copaiba balsam oil; coriander oil; costus_root oil; cumin oil; cypress oil; davana oil; dill oil; dillseed oil; eau de brouts absolute; oakmoss absolute; elemi oil; tarragon oil; eucalyptus citriodora oil; eucalyptus oil; fennel oil; fir oil; galbanum oil; galbanum resin; geranium oil; grapefruit oil; guaiac wood oil; gurjun balsam; gurjun balsam oil; helichrysum absolute; helichrysum oil; ginger oil; iris root abolute; iris root oil; jasmine absolute; calamus oil; blue camomile oil; Roman camomile oil; carrot-seed oil; cascarilla oil; pine-needle oil; spearmint oil; caraway oil; labdanum oil; labdanum absolute; labdanum resin; lavandin absolute; lavandin oil; lavender absolute; lavender oil; lemongrass oil; lovage oil; distilled lime oil; pressed lime oil; linaloe oil; litsea cubeba oil; bay-leaf oil; mace oil; marjoram oil; mandarin oil; massoi bark oil; mimosa absolute; ambrette oil; tincture of musk; clary sage Oil; myristica oil; myrrh absolute; myrrh oil; myrtle oil; clove leaf oil; clove flower oil; neroli oil; olibanum abolute; olibanum oil; opopanax oil; orange blossom absolute; orange oil; origanum oil; palmarosa oil; patchouli oil; perilla oil; Peru balsam oil; parsley leaf oil; parsley seed oil; petitgrain oil; peppermint oil; pepper oil; pimento oil; pine oil; pennyroyal oil; rose absolute; rosewood oil; rose oil; rosemary oil; Dalmatian sage oil; Spanish sage oil; sandalwood oil; celery seed oil; spike lavender oil; Japanese aniseed oil; styrax oil; tagetes oil; fir-needle oil; tea-tree oil; turpentine oil; thyme oil; Tolu balsam; tonka absolute; tuberose absolute; vanilla extract; violet leaf absolute; verbena oil; vetiver oil; juniper oil; wine-lees oil; wormwood oil; wintergreen oil; ylang oil; hyssop oil; civet absolute; cinnamon leaf oil; cinnamon bark oil; as well as fractions thereof or constituents isolated therefrom;

Preferred individual fragrance materials are selected from the group consisting of:
Hydrocarbons, such as for example 3-carene; α-pinene; β-pinene; α-terpinene; γ-terpinene; p-cymene; bisabolene; camphene; caryophyllene, cedrene; farnesene; liminene; longifolene; myrcene; ocimene; valencene; (E,Z)-1,3,5-undecatriene;
Aliphatic alcohols such as for example hexanol; octanol; 3-octanol; 2,6-dimethylheptanol; 2-methylheptanol; 2-methyloctanol; (E)-3-hexenol; (E) and (Z)-3-hexenol; 1-octen-3-ol; mixtures of 3,4,5,6,6-pentamethyl-3/4-hepten-2-ol and 3,5,6,6-tetramethyl-4-methyleneheptan-2-ol; (E,Z)-2,6-nonadienol; 3,7-dimethyl-7-methoxyoctan-2-ol; 9-decenol; 10-undecenol; 4-methyl-3-decen-5-ol; aliphatic aldehydes and their acetals such as for example hexanal; heptanal; octanal; nonanal; decanal; undecanal; dodecanal; tridecanal; 2-methyloctanal; 2-methylnonanal; (E)-2-hexenal; (Z)-4-heptenal; 2,6-dimethyl-5-heptenal; 10-undecenal; (E)-4-decenal; 2-dodecenal; 2,6,10-trimethyl-5,9-undecadienal; heptanal diethyl acetal; 1,1-dimethoxy-2,2,5-trimethyl-4-hexene; citronellyl oxyacetaldehyde;
Aliphatic ketones and oximes thereof such as for example 2-heptanone; 2-octanone; 3-octanone; 2-nonanone; 5-methyl-3-heptanone; 5-methyl-3-heptanone oxime; 2,4,4,7-tetramethyl-6-octen-3-one; aliphatic sulphur-containing compounds such as for example
   3-methylthiohexanol; 3-methylthiohexyl acetate; 3-mercaptohexanol; 3-mercaptohexyl acetate; 3-mercaptohexyl butyrate; 3-acetylthiohexyl acetate; 1-menthene-8-thiol;
Aliphatic nitriles such as for example 2-nonenenitrile; 2-tridecenenenitrile; 2,12-tridecenene-nitrile; 3,7-dimethyl-2,6-octadienenitrile; 3,7-dimethyl-6-octenenitrile;
Aliphatic carboxylic acids and esters thereof, such as for example (E)- and (Z)-3-hexenyl formate; ethyl acetoacetate; isoamyl acetate; hexyl acetate; 3,5,5-trimethylhexyl acetate; 3-methyl-2-butenyl acetate; (E)-2-hexenyl acetate; (E)- and (Z)-3-hexenyl acetate; octyl acetate; 3-octyl acetate; 1-octen-3-yl acetate; ethyl butyrate; butyl butyrate; isoamyl butyrate; hexyl butyrate; (E)- and (Z)-3-hexenyl isobutyrate; hexyl crotonate; ethyl isovalerate; ethyl 2-methylpentanoate; ethyl hexanoate; allyl hexanoate; ethyl heptanoate; allyl heptanoate; ethyl octanoate; ethyl (E,Z)-2,4-decadienoate; methyl 2-octynoate; methyl 2-nonynoate; allyl-2-isoamyloxyacetate; methyl-3,7-dimethyl-2,6-octadienoate;
Acyclic terpene alcohols such as for example citronellol; geraniol; nerol; linalool; lavandulol; nerolidol; farnesol; tetrahydrolinalool; tetrahydrogeraniol; 2,6-dimethyl-7-octen-2-ol; 2,6-dimethyloctan-2-ol; 2-methyl-6-methylene-7-octen-2-ol; 2,6-dimethyl-5,7-octadien-2-ol; 2,6-dimethyl-3,5-octadien-2-ol; 3,7-dimethyl-4,6-octadien-3-ol; 3,7-dimethyl-1,5,7-octatrien-3-ol; 2,6-dimethyl-2,5,7-octatrien-1-ol; and formates, acetates, propionates, isobutyrates, butyrates, isovalerates, pentanoates, hexanoates, crotonates, tiglinates, 3-methyl-2-butenoates thereof;
Acyclic terpene aldehydes and ketones such as for example geranial; neral; citronellal;
   7-hydroxy-3,7-dimethyloctanal; 7-methoxy-3,7-dimethyloctanal; 2,6,10-trimethyl-9-undecenal; geranylacetone; and the dimethyl and diethyl acetals of geranial, neral, 7-hydroxy-3,7-dimethyloctanal;
Cyclic terpene alcohols such as for example menthol; isopulegol; alpha-terpineol; terpineol-4; menthan-8-ol; menthan-1-ol; menthan-7-ol; borneol; isoborneol; linalool oxide; nopol; cedrol; ambrinol; vetiverol; guaiol; and the formates, acetates, propionates, isobutyrates, butyrates, isovalerates, pentanoates, hexanoates, crotonates, tiglinates, 3-methyl-2-butenoates thereof;
Cyclic terpene aldehydes and ketones such as for example menthone; isomenthone;
   8-mercaptomenthan-3-one; carvone; camphor; fenchone; alpha-ionone; beta-ionone; alpha-n-methylionone; beta-n-methylionone; alpha-isomethylionone; beta-isomethylionone; alpha-irone; alpha-damascone; beta-damascone; beta-damascenone; delta-damascone; gamma-damascone; 1-(2,4,4-trimethyl-2-cyclohexen-1-yl)-2-buten-1-one; 1,3,4,6,7,8a-hexahydro-1,1,5,5-tetramethyl-2H-2,4-a-methanonaphthalen-8(5H)-one; nootkatone; dihydronootkatone; alpha-sinensal; beta-sinensal; acetylated cedarwood oil (methyl cedryl ketone);
Cyclic alcohols such as for example 4-tert.-butylcyclohexanol; 3,3,5-trimethylcyclohexanol; 3-isocamphylcyclohexanol; 2,6,9-trimethyl-Z2,Z5,E9-cyclododecatrien-1-ol; 2-isobutyl-4-methyltetrahydro-2H-pyran-4-ol;
Cycloaliphatic alcohols such as for example alpha-3,3-trimethylcyclohexylmethanol;
   2-methyl-4-(2,2,3-trimethyl-3-cyclopent-1-yl)butanol; 2-methyl-4-(2,2,3-trimethyl-3-cyclopent-1-yl)-2-buten-1-ol; 2-ethyl-4-(2,2,3-trimethyl-3-cyclopent-1-yl)-2-buten-1-ol;
   3-methyl-5-(2,2,3-trimethyl-3-cyclopent-1-yl)-pentan-2-ol; 3-methyl-5-(2,2,3-trimethyl-3-cyclopent-1-yl)-4-penten-2-ol; 3,3-dimethyl-5-(2,2,3-trimethyl-3-cyclopent-1-yl)-4-penten-2-ol; 1-(2,2,6-trimethylcyclohexyl)pentan-3-ol; 1-(2,2,6-trimethylcyclohexyl)hexan-3-ol;
Cyclic and cycloaliphatic ethers such as for example cineol; cedryl methyl ether; cyclododecyl methyl ether; (ethoxymethoxy)cyclododecane; alpha-cedrene epoxide; 3a,6,6,9a-tetramethyl-dodecahydronaphtho[2,1-b]furan; 3a-ethyl-6,6,9a-trimethyldodecahydro-naphtho[2,1-b]furan; 1,5,9-trimethyl-13-oxabicyclo[10.1.0]trideca-4,8-diene; rose oxide; 2-(2,4-dimethyl-3-cyclohexen-1-yl)-5-methyl-5-(1-methylpropyl)-1,3-dioxane;
Cyclic ketones such as for example 4-tert.-butylcyclohexanone; 2,2,5-trimethyl-5-pentylcyclopentanone; 2-heptylcyclopentanone; 2-pentylcyclopentanone; 2-hydroxy-3-methyl-2-cyclopenten-1-one; 3-methyl-cis-2-penten-1-yl-2-cyclopenten-1-one; 3-methyl-2-pentyl-2-cyclopenten-1-one; 3-methyl-4-cyclopentadecenone; 3-methyl-5-cyclopentadecenone; 3-methylcyclopentadecanone; 4-(1-ethoxyvinyl)-3,3,5,5-tetramethylcyclohexanone; 4-tert.-pentylcyclohexanone; 5-cyclohexadecen-1-one; 7-cyclohexadecen-1-one; 8-cyclohexadecen-1-one; 6,7-dihydro-1,1,2,3,3-pentamethyl-4(5H)-indanone; 9-cycloheptadecen-1-one; cyclopentadecanone; cyclohexadecanone;
Cycloaliphatic aldehydes such as for example 2,4-dimethyl-3-cyclohexenecarbaldehyde; 2-methyl-4-(2,2,6-trimethyl-cyclohexen-1-yl)-2-butenal; 4-(4-hydroxy-4-methylpentyl)-3-cyclohexenecarbaldehyde; 4-(4-methyl-3-penten-1-yl)-3-cyclohexenecarbaldehyde;
Cycloaliphatic ketones such as for example 1-(3,3-dimethylcyclohexyl)-4-penten-1-one;
   1-(5,5-dimethyl-1-cyclohexen-1-yl)-4-penten-1-one; 2,3,8,8-tetra methyl-1,2,3,4,5,6,7,8-octahydro-2-naphthalenyl methyl ketone; methyl-2,6,10-trimethyl-2,5,9-cyclododecatrienyl ketone; tert.-butyl (2,4-dimethyl-3-cyclohexen-1-yl)ketone;
Esters of cyclic alcohols such as for example 2-tert.-butylcyclohexyl acetate; 4-tert.-butyl-cyclohexyl acetate; 2-tert.-pentylcyclohexyl acetate; 4-tert.-pentylcyclohexyl acetate; decahydro-2-naphthyl acetate; 3-pentyltetrahydro-2H-pyran-4-yl acetate; decahydro-2,5,5,8a-tetramethyl-2-naphthyl acetate; 4,7-methano-3a,4,5,6,7,7a-hexahydro-5 or 6-indenyl acetate; 4,7-methano-3a,4,5,6,7,7a-hexahydro-5 or 6-indenyl propionate; 4,7-methano-3a,4,5,6,7,7a-hexahydro-5 or 6-indenyl isobutyrate; 4,7-methanooctahydro-5 or 6-indenyl acetate;
Esters of cycloaliphatic carboxylic acids such as for example, allyl 3-cyclohexylpropionate; allyl cyclohexyloxyacetate; methyl dihydrojasmonate; methyl jasmonate; methyl 2-hexyl-3-oxocyclopentanecarboxylate; ethyl 2-ethyl-6,6-dimethyl-2-cyclohexenecarboxylate; ethyl 2,3,6,6-tetramethyl-2-cyclohexenecarboxylate; ethyl 2-methyl-1,3-dioxolane-2-acetate;
Aromatic hydrocarbons such as for example styrene and diphenylmethane;
Araliphatic alcohols such as for example benzyl alcohol; 1-phenylethyl alcohol; 2-phenylethyl alcohol; 3-phenylpropanol; 2-phenylpropanol; 2-phenoxyethanol; 2,2-dimethyl-3-phenylpropanol; 2,2-dimethyl-3-(3-methylphenyl)propanol; 1,1-di methyl-2-phenylethyl alcohol; 1,1-dimethyl-3-phenylpropanol; 1-ethyl-1-methyl-3-phenylpropanol; 2-methyl-5-phenylpentanol; 3-methyl-5-phenylpentanol; 3-phenyl-2-propen-1-ol; 4-methoxybenzyl alcohol; 1-(4-isopropylphenyl)ethanol;
Esters of araliphatic alcohols and aliphatic carboxylic acids such as for example benzyl acetate; benzyl propionate; benzyl isobutyrate; benzyl isovalerate; 2-phenylethyl acetate;
   2-phenylethyl propionate; 2-phenylethyl isobutyrate; 2-phenylethyl isovalerate; 1-phenylethyl acetate; alpha-trichloromethylbenzyl acetate; alpha,alpha-dimethylphenylethyl acetate; alpha,alpha-dimethylphenylethyl butyrate; cinnamyl acetate; 2-phenoxyethyl isobutyrate; 4-methoxybenzyl acetate; araliphatic ethers such as for example 2-phenylethyl methyl ether;
   2-phenylethyl isoamyl ether; 2-phenylethyl 1-ethoxyethyl ether; phenylacetaldehyde dimethyl acetal; phenylacetaldehyde diethyl acetal; hydratropaldehyde dimethyl acetal; phenylacetaldehyde glycerol acetal; 2,4,6-trimethyl-4-phenyl-1,3-dioxane; 4,4a,5,9b-tetrahydroindeno[1,2-d]-m-dioxin; 4,4a,5,9b-tetrahydro-2,4-dimethylindeno[1,2-d]-m-dioxin;
Aromatic and araliphatic aldehydes such as for example benzaldehyde; phenylacetaldehyde; 3-phenylpropanal; hydratropaldehyde; 4-methylbenzaldehyde; 4-methylphenylacetaldehyde; 3-(4-ethylphenyl)-2,2-dimethylpropanal; 2-methyl-3-(4-isopropylphenyl)propanal; 2-methyl-3-(4-tert.-butylphenyl)propanal; 3-(4-tert.-butylphenyl)propanal; cinnamaldehyde; alpha-butylcinnamaldehyde; alpha-amylcinnamaldehyde; alpha-hexylcinnamaldehyde; 3-methyl-5-phenylpentanal; 4-methoxybenzaldehyde; 4-hydroxy-3-methoxybenzaldehyde; 4-hydroxy-3-ethoxybenzaldehyde; 3,4-methylenedioxybenzaldehyde; 3,4-dimethoxy-benzaldehyde; 2-methyl-3-(4-methoxyphenyl)propanal; 2-methyl-3-(4-methylenedioxyphenyl)propanal;
Aromatic and araliphatic ketones such as for example acetophenone; 4-methylacetophenone; 4-methoxyacetophenone; 4-tert.-butyl-2,6-dimethylacetophenone; 4-phenyl-2-butanone; 4-(4-hydroxyphenyl)-2-butanone; 1-(2-naphthalenyl)ethanone; benzophenone; 1,1,2,3,3,6-hexamethyl-5-indanyl methyl ketone; 6-tert.-butyl-1,1-dimethyl-4-indanyl methyl ketone; 1-[2,3-dihydro-1,1,2,6-tetramethyl-3-(1-methylethyl)-1H-5-indenyl]ethanone; 5′,6′,7′,8′-tetrahydro-3′,5′,5′,6′,8′,8′-hexamethyl-2-acetonaphthone;
Aromatic and araliphatic carboxylic acids and esters thereof such as for example benzoic acid; phenylacetic acid; methyl benzoate; ethyl benzoate; hexyl benzoate; benzyl benzoate; methyl phenylacetate; ethyl phenylacetate; geranyl phenylacetate; phenylethyl phenylacetate; methyl cinnamate; ethyl cinnamate; benzyl cinnamate; phenylethyl cinnamate; cinnamyl cinnamate; allyl phenoxyacetate; methyl salicylate; isoamyl salicylate; hexyl salicylate; cyclohexyl salicylate; cis-3-hexenyl salicylate; benzyl salicylate; phenylethyl salicylate; methyl 2,4-dihydroxy-3,6-dimethylbenzoate; ethyl 3-phenylglycidate; ethyl 3-methyl-3-phenylglycidate;
Nitrogen-containing aromatic compounds such as for example 2,4,6-trinitro-1,3-dimethyl-5-tert.-butylbenzene; 3,5-dinitro-2,6-dimethyl-4-tert.-butylacetophenone; cinnamonitrile; 5-phenyl-3-methyl-2-pentenenitrile; 5-phenyl-3-methylpentanenitrile; methyl anthranilate; methyl N-methylanthranilate; Schiff bases of methyl anthranilate with 7-hydroxy-3,7-dimethyloctanal; 2-methyl-3-(4-tert.-butylphenyl)propanal or 2,4-dimethyl-3-cyclohexene-carbaldehyde; 6-isopropylquinoline; 6-isobutylquinoline; 6-sec.-butylquinoline; indole; skatole; 2-methoxy-3-isopropylpyrazine; 2-isobutyl-3-methoxypyrazine;
Phenols, phenyl ethers and phenyl esters such as for example estragole; anethole; eugenol; eugenyl methyl ether; isoeugenol; isoeugenyl methyl ether; thymol; carvacrol; diphenyl ether; beta-naphthyl methyl ether; beta-naphthyl ethyl ether; beta-naphthyl isobutyl ether; 1,4-dimethoxybenzene; eugenyl acetate; 2-methoxy-4-methylphenol; 2-ethoxy-5-(1-propenyl)phenol; p-cresyl phenylacetate;
Heterocyclic compounds such as for example 2,5-dimethyl-4-hydroxy-2H-furan-3-one;
   2-ethyl-4-hydroxy-5-methyl-2H-furan-3-one; 3-hydroxy-2-methyl-4H-pyran-4-one; 2-ethyl-3-hydroxy-4H-pyran-4-one;
Lactones such as for example 1,4-octanolide; 3-methyl-1,4-octanolide; 1,4-nonanolide;
   1,4-decanolide; 8-decen-1,4-olide; 1,4-undecanolide; 1,4-dodecanolide; 1,5-decanolide;
   1,5-dodecanolide; 1,15-pentadecanolide; cis and trans-1′-pentadecen-1,15-olide; cis and trans-12-pentadecen-1,15-olide; 1,16-hexadecanolide; 9-hexadecen-1,16-olide; 10-oxa-1,16-hexadecanolide; 11-oxa-1,16-hexadecanolide; 12-oxa-1,16-hexadecanolide; ethylene 1,12-dodecanedioate; ethylene 1,13-tridecanedioate; coumarin; 2,3-dihydrocoumarin; octahydrocoumarin.

Perfumes used in the invention can be used in a liquid form, undiluted or diluted, where the fragrance materials are mixed with a solvent (diluent). Preferred is a solid air freshener according to the invention, wherein the solvent is selected from the group consisting of ethanol, isopropanol, diethylene glycol monoethyl ether, glycerol, propylene glycol, 1,2-butylene glycol, dipropylene glycol, diethyl phthalate, triethyl citrate, isopropyl myristate, and mixtures thereof.

The concept of aromatherapy can be interesting in the context of the present invention, and therefore aroma therapy fragrance materials are preferred in component (iii) but fragrances not encompassing aroma-therapeutic properties are also useful for air freshening purposes.

Examples for fragrance materials or perfumes with aroma-therapeutic properties that can be used in the invention are menthol, lavender, camphor, rosemary, eucalyptus, citronella, clove, jasmine, lemongrass, patchouli, peppermint, sandalwood etc.

### Optional Ingredients (Component v))

Further components (optional ingredients) which can be present in the air freshener according to the invention give rise to additional effects. Examples which may be mentioned are:

preservatives, abrasives, antibacterial agents, anti-inflammatory agents, anti-irritants, irritation suppressants, antimicrobial agents, antioxidants, antiseptics, binders, buffers, chelating agents, deodorisers, emollients, fixatives, foaming agents, foam stabilisers, antifoams, foam boosters, fungicides, humectants, moisturisers, bleaching agents, stain removers, soil repellents, insect repellents, opacifiers, plasticisers, brighteners, abrasive agents, skin care agents, skin-protecting agents, skin-softening agents, skin-cooling agents, skin-warming agents, stabilisers, UV-absorbers, thickeners, vitamins, oils, waxes, fats, phospholipids, mono- or polyunsaturated fatty acids, α-hydroxy acids, polyhydroxy fatty acids, dyes, color-protection agents, pigments, plant extracts, electrolytes, organic solvents or silicone derivatives.

In case any optional ingredient can be assigned to one or more of components (i), (ii) and (iii) it is assigned to the first of the mentioned components to which it can be assigned. As stated above, for the calculations mentioned water cannot be considered as an optional ingredient of component (v).

Optional ingredients such as chelating agents and salts thereof (e.g. EDTA, disodium EDTA and tetrasodium EDTA or citric acid) and/or opacifiers (metal oxides like e.g. titanium dioxide) are preferably part of the air fresheners according to the present invention. Chelating agents such as EDTA are commercially available.

The solid air fresheners of the present invention may also comprise agents for adjusting a pH value or the ionic strength. Examples which may be mentioned are phosphoric acid and salts thereof, sodium acetate, acetic acid, citric acid and salts thereof, EDTA, disodium EDTA and tetrasodium EDTA.

The air fresheners of present invention may also comprise electrolytes, minerals or salts such as sodium chloride (typically up to 0.5 wt. %).

Preferred is a solid air freshener according to the invention, comprising

(v) a total amount of from 0 to 5 wt.-% further components (optional ingredients), preferably 0.5 to 2 wt.-% further components,
wherein the total amount of components (i), (ii), (iii), (iv), and (v) is 100 wt.-%, based on the total weight of the solid air freshener.

A further aspect of the present invention relates to a process of manufacturing a solid air freshener according the invention, in particular in a preferred embodiment, comprising the following steps:

a) providing a castable or pourable mixture comprising components (i), (ii), and (iii) and, optionally, components (iv) and/or (v)
b) introducing the mixture obtained in step a) into a mould;
c) solidifying said mixture in said mould, and
d) removing the resulting solid air freshener from said mould.

Preferred is a process of manufacturing a solid air freshener, wherein components (i) and (ii) are mixed in the solid state, and the mixture is heated to obtain a pourable melt, in the absence of components (iii), (iv), and (v).

In alternative preferred processes of the present invention, for manufacturing air fresheners (a) the components i) and ii) are melted separately and then mixed, (b) component i) is melted and component ii) in solid form is added and the mixture is melted and mixed together, or (c) component ii) is melted and component i) in solid form is added and the mixture is melted and mixed together. Thereto component iii) and optionally component iv) and/or component v), are added. The resulting molten mass comprising components i), ii), iii), and optionally component iv) and/or v) is subsequently poured or cast into a mould (step b) and cooled in said mould to room temperature (20° C.). To aid the cooling process the moulds may be placed in a tray of cold water. When the melt has solidified (hardened), the resulting air freshener is removed from the mould.

Preferably, the components i) and ii) are melted at a temperature of up to 85° C., preferably at a temperature of up to 75° C., most preferably in the range of 60-70° C. Component iii) is added thereto and dissolved in the mixture of components i) and ii).

Further ingredients (as stated above) may be incorporated into the mixture of compounds i), ii) and iii) prior to casting the mixture into moulds.

The cast moulds may be allowed to cool at room temperature, or cooling is accelerated by placing the moulds in a tray of cold water. The cast air fresheners can usually be removed from the moulds within 2 hours of casting.

Prolonged heating of the mixture to temperatures exceeding 85° C. should be avoided.

The air fresheners of the present invention may be of any size or shape. Their shape for example may be round, oval, rectangular, quadratic, spherical, ellipsoidal, cuboid, helical or irregularly shaped, e.g. in the form of figures like crocodiles, cats, horses, ducks, roses, sunflowers, palm trees, shells, fish, whales, dolphins, ships, stars, starfish.

A further aspect of the present invention relates to a method of releasing a fragrance from a solid air freshener according to the invention or manufactured according to the process according to the invention, comprising the following step:

(m1) dissolving a portion of the solid air freshener in water to obtain an aqueous solution,
(m2) exposing the aqueous solution obtained in step (m1) to air so that fragrance is released into said air.

Preferred is a method according to the invention, wherein in step (m2) or in an additional step the aqueous solution obtained in step (m1) is allowed to flow toward a sink or drainage system while being exposed to air so that fragrance is released into said air.

The present invention also relates to the use of a solid air freshener according to the invention, in particular in one of the preferred embodiments, for controlled release of fragrance into the air. Preferred is a use according to the invention for controlled release of fragrance into the air contained in a bathroom.

Another aspect of the present invention relates to a sanitary or bathroom appliance comprising a receptacle containing a solid air freshener according to the invention, in particular according to one of the preferred embodiments, wherein the receptacle comprises one or more water inlets and one or more discharge outlets for an aqueous solution of the air freshener.

The invention is explained further with the aid of the following examples, but not limited thereto. Unless stated otherwise, the data relate to the weight.

### Examples 1 and 2

| | | Ex. 1 | Ex. 2 | |
|---|---|---|---|---|
| | | (parts | (parts | |
| | | by | by | |
| | Supplier | weight) | weight) | Remarks |
| Pluriol ® E 9000 | BASF | 40 | 40 | Part A |
| (Polyethylene Glycol, | | | | (60° C.- |
| M.W. 9000) | | | | 70° C.) |
| Pluriol ® E 1500 | BASF | 10 | — | |
| (Polyethylene Glycol, | | | | |
| M.W. 1500) | | | | |
| Eumulgin ® B3 | Cognis | 10 | 10 | |
| (Ceteareth-30) | | | | |
| Mentholated Lavender | Symrise | 40 | 50 | Part B |
| fragrance | | | | |
| Total | | 100 | 100 | |

### Procedure:

1. Melt the ingredients of Part A. Mix well.
2. Add the ingredients of Part B. Mix well.
3. Transfer mixture into moulds.
4. Once mixture has cooled and hardened into shape, remove from moulds.

In examples 1 and 2, a solid air freshener article is formed. However, the rate of solidification is slower in example 2 as the amount of polyethylene glycol is reduced and the fragrance load is increased.

### Examples 3 and 4

| | | Ex. 3 | Ex. 4 | |
|---|---|---|---|---|
| | | (parts | (parts | |
| | | by | by | |
| | Supplier | weight) | weight) | Remarks |
| Pluriol ® E 9000 | BASF | 64.5 | 29.46 | Part A |
| (Polyethylene Glycol, | | | | (60° C.- |
| M.W. 9000) | | | | 70° C.) |
| Pluriol ® E 1500 | BASF | — | 30 | |
| (Polyethylene Glycol, | | | | |
| M.W. 1500) | | | | |
| Eumulgin ® B3 | Cognis | 10 | 15 | |
| (Ceteareth-30, 100%) | | | | |
| Incrosoft ® T-90 HF | Croda | 5 | 5 | |
| (Polyoxyethylene Ditallow | | | | |
| Diamido Methosulfate, 90%; | | | | |
| Propylene Glycol 10%) | | | | |
| Mentholated Lavender | Symrise | 20 | 20 | Part B |
| fragrance | | | | |
| Jaguar C13-S | Rhodia | 0.5 | 0.5 | |
| (Guar Hydroxypropyl- | | | | |
| trimonium Chloride) | | | | |
| 5/002470 Turquoise (1%) | Symrise | — | 0.04 | |
| (Color dye) | | | | |
| Total | | 100 | 100 | |

### Procedure:

1. Melt the ingredients of Part A. Mix well.
2. Add the ingredients of Part B accordingly. Mix well.
3. Transfer mixture into moulds.
4. Once mixture has cooled and hardened into shape, remove from moulds.

In examples 3 and 4, a solid air freshener article is formed. Both examples show a good solidification and example 4 has a higher degree of water solubility than example 3 because of the higher amount of surfactants and the lower amount of polyethylene glycol with a molecular weight of 9000.

As the articles are cast from moulds, different shapes and sizes can be produced.

### Examples 5, 6 and 7

### In-Use Evaluation

| | | Ex. 5 | Ex. 6 | Ex. 7 | |
|---|---|---|---|---|---|
| | | (parts | (parts | (parts | |
| | | by | by | by | |
| | Supplier | weight) | weight) | weight) | Remarks |
| Pluriol ® E 9000 | BASF | 20 | 20 | 20 | Part A |
| (Polyethylene Glycol, | | | | | (60° C.- |
| M.W. 9000) | | | | | 70° C.) |
| Pluriol ® E 1500 | BASF | 30 | 30 | 30 | |
| (Polyethylene Glycol, | | | | | |
| M.W. 1500) | | | | | |
| Eumulgin ® B3 | Cognis | 45 | 40 | 30 | |
| (Ceteareth-30) | | | | | |
| Mentholated Lavender | Symrise | 5 | 10 | 20 | Part B |
| fragrance | | | | | |
| Total | | 100 | 100 | 100 | |

### Procedure:

1. Melt the ingredients of Part A. Mix well.
2. Add the ingredients of Part B. Mix well.
3. Transfer mixture into moulds.
4. Once mixture has cooled and hardened into shape, remove from moulds.

### Evaluation Protocol

a) 10 ml of water is poured over each sample (formulations 5, 6 and 7; sample weight 25 g each) and collected in a Petri-dish.
b) The Petri-dish is placed in an evaluation booth measuring 2 m (height)×0.89 m (width)×0.97 m (depth) having an individual air-circulation system. The intensity of the fragrance in the booth is evaluated by a panel of experts at predetermined intervals (5 minutes, 1 hour and 6 hours)

### Results (Average Scores of 10 Panelists)

| | | Sample 5 | Sample 6 | Sample 7 |
|---|---|---|---|---|
| 5 | minutes | 1 | 4 | 5 |
| 1 | hour | 1 | 3 | 5 |
| 6 | hours | 1 | 1 | 4 |

Score (Intensity): 1 (weak)-5 (strong)

The results show that sample 6 and sample 7 will give a perceivable burst of fragrance in the immediate stage. This is important as the consumer can appreciate the presence of the fragrance when he or she is having the shower. Sample 7 with a fragrance dosage of 20% yields particularly good results in the initial stage and the presence of the fragrance in the cubicle remains perceivable for at least 6 hours.

1. Solid air freshener comprising the following components:
i) a total amount of polyethylene glycols in the range of from 30 to 80 wt.-%, wherein the weight average molecular weight M_{W} of the total amount of polyethylene glycols present in the solid air freshener is in the range of from 1000 to 20000 g/mol, the total amount of polyethylene glycols consisting of one, two, or more polyethylene glycol materials,
ii) a total amount of surfactants in the range of from 5 to 40 wt.-%,
iii) a total amount of perfume in the range of from 5 to 50 wt.-%, wherein the perfume consists of
(iii)(a) fragrance material, and optionally
(iii)(b) a solvent for the fragrance material,
iv) an amount of water in the range of from 0 to 10 wt.-%,
wherein any water present in the solid air freshener is exclusively considered as component (iv), and
wherein the total amount of components (i), (ii), (iii), and (iv) is at least 95 wt.-%, based on the total weight of the solid air freshener.

2. Solid air freshener according to claim 1, wherein the solvent is selected from the group consisting of ethanol, isopropanol, diethylene glycol monoethyl ether, glycerol, propylene glycol, 1,2-butylene glycol, dipropylene glycol, diethyl phthalate, triethyl citrate, isopropyl myristate, and mixtures thereof.

3. Solid air freshener according to claim 1, wherein
component i) consists of two or more polyethylene glycol materials, each of said materials having a weight average molecular weight M_{W} in the range of from 1000 to 20000, wherein the difference between the respective weight average molecular weights of the two or more polyethylene glycol materials is at least 2000.

4. Solid air freshener according to claim 1, wherein component i) consists of
(i)(a) a first polyethylene glycol material having a weight average molecular weight M_{W(i)(a)} in the range of from 1500 to 3000, and
(i)(b) a second polyethylene glycol material having a weight average molecular weight M_{W(i)(b)} in the range of from 5500 to 9500, and
optionally one or more further polyethylene glycol materials having a different weight average molecular weight M_{W}.

5. Solid air freshener according to claim 4,
wherein the ratio (TMWFL Index)
[(M_{W(i)(a)}×weight percentage of component (i)(a))+(M_{W(i)(b)}×weight percentage of component (i)(b))]:[(weight percentage of component (iii)(a))×g/mol]
is greater than 7000.

6. Solid air freshener according to claim 5, wherein said ratio is in the range of from 8000 to 25000.

7. Solid air freshener according to claim 1, wherein component i) consists of a single polyethylene glycol material having a weight average molecular weight M_{W} in the range of from 4000 to 9000.

8. Solid air freshener according to claim 7, wherein the ratio (TMWFL Index)
[(M_{W(i)}×weight percentage of component (i)]:[(weight percentage of component (iii)(a))×g/mol]
is greater than 700.

9. Solid air freshener according to claim 1, wherein
(i) the total amount of polyethylene glycols is in the range of from 40 to 70 wt.-%, and/or
(ii) the total amount of surfactants in the range of from 10 to 30 wt.-%, and/or
(iii) the total amount of perfume is in the range of from 10 to 30 wt.-%, and/or
(iv) the amount of water is in the range of from 0 to 5 wt.-%,
wherein the total amount of components (i), (ii), (iii), and (iv) is at least 95 wt.-%, based on the total weight of the solid air freshener.

10. Solid air freshener according to claim 3, wherein
(i) the total amount of polyethylene glycols is in the range of from 40 to 70 wt.-%, and
(ii) the total amount of surfactants in the range of from 10 to 30 wt.-%, and
(iii) the total amount of perfume is in the range of from 10 to 30 wt.-%, and
(iv) the amount of water is in the range of from 0 to 5 wt.-%,
wherein the total amount of components (i), (ii), (iii), and (iv) is at least 95 wt.-%, based on the total weight of the solid air freshener.

11. Solid air freshener according to claim 9, comprising
(v) a total amount of from 0 to 5 wt.-% further components
wherein the total amount of components (i), (ii), (iii), (iv), and (v) is 100 wt.-%, based on the total weight of the solid air freshener.

12. Process of manufacturing a solid air freshener of claim 11, comprising:
a) providing a castable or pourable mixture comprising components (i), (ii), and (iii) and, optionally, components (iv) and/or (v);
b) introducing the mixture obtained in step a) into a mould;
c) solidifying said mixture in said mould; and
d) removing the resulting solid air freshener from said mould.

13. Process according to claim 12, wherein components (i) and (ii) are mixed in the solid state, and the mixture is heated to obtain a pourable melt, in the absence of components (iii), (iv), and (v).

14. Method of releasing a fragrance from a solid air freshener of claim 1, comprising:
(m1) dissolving a portion of the solid air freshener in water to obtain an aqueous solution,
(m2) exposing the aqueous solution obtained in step (m1) to air so that fragrance is released into said air.

15. Method according to claim 14, wherein in step (m2) or in an additional step the aqueous solution obtained in step (m1) is allowed to flow toward a sink or drainage system while being exposed to air so that fragrance is released into said air.

16. Method for the controlled release of fragrance into the air comprising exposing the solid air freshener of claim 1 to a humid environment so that fragrance is released.

17. Method according to claim 16, wherein the humid environment is in a bathroom.

18. Sanitary or bathroom appliance comprising a receptacle containing a solid air freshener of claim 1, wherein the receptacle comprises one or more water inlets and one or more discharge outlets for an aqueous solution of the air freshener.

19. Solid air freshener according to claim 1, wherein the perfume consists of
(iii)(a) fragrance material, and
(iii)(b) a solvent for the fragrance material.

20. Solid air freshener according to claim 19, wherein the solvent is selected from the group consisting of ethanol, isopropanol, diethylene glycol monoethyl ether, glycerol, propylene glycol, 1,2-butylene glycol, dipropylene glycol, diethyl phthalate, triethyl citrate, isopropyl myristate, and mixtures thereof.
